(19) United States
(12) Patent Application Publication
(10) Pub. No.: US 2008/0138390 A1
(43) Pub. Date: Jun. 12, 2008

(54) Transdermal Delivery System For Use With Basic Permeation Enhancers

(76) Inventors: Tsung-Min Hsu, Minneapolis, MN (US); Eric C. Jacobson, San Diego, CA (US); Eric C. Luo, Plano, TX (US); Alan T.J. Hickey, Eden Prairie, MN (US); Nicole T. Gricenko, St. Louis Park, MN (US)

Correspondence Address: INTELLECTUAL PROPERTY GROUP;FREDRIKSON & BYRON, P.A., 200 SOUTH SIXTH STREET, SUITE 4000, MINNEAPOLIS, MN, 55402, US

(21) Appl. No.: 11/547,987

(22) PCT Filed: Apr. 7, 2005
(86) PCT No.: PCT/US05/12163

§ 371 Date: Aug. 17, 2007

Related U.S. Application Data
(60) Provisional application No. 60/560,499, filed on Apr. 7, 2004.

(51) Int. Cl. A61K 9/70

(52) U.S. Cl. 424/449

(57) ABSTRACT

The present invention includes transdermal delivery systems having a polymeric active agent reservoir fabricated from an admixture of polyisobutylene and an insoluble hydrophilic polymer in powdered form, which provide numerous advantages in the transdermal delivery of active agents using basic enhancer compositions. For example, the systems of the invention provide for (1) increased permeation of the active agent through the skin, (2) an improved capability of extracting the active agent and enhancer from the transdermal systems, (3) enhanced structural integrity, (4) good chemical stability, (5) reduced phase separation, and (6) decreased cold flow.

### TECHNICAL FIELD

This invention relates generally to delivery systems for the topical and transdermal administration of pharmacologically active agents using basic permeation enhancers, and more specifically relates to a monolithic transdermal delivery system that is particularly adapted to use with basic permeation enhancers such as inorganic hydroxides and the like.

### BACKGROUND ART

The delivery of active agents through the skin provides many advantages; primarily, such a means of delivery is a comfortable, convenient, arid noninvasive way of administering active agents. The variable rates of absorption and metabolism encountered in oral treatment are avoided, and other inherent inconveniences, e.g., gastrointestinal irritation and the like, are eliminated as well. Transdermal active agent delivery also makes possible a high degree of control over blood concentrations of any particular active agent.

Skin is a structurally complex, relatively thick membrane. Molecules moving from the environment into and through intact skin must first penetrate the stratum corneum and any material on its surface. They must then penetrate the viable epidermis, the papillary dermis, and the capillary walls into the blood stream or lymph channels. To be so absorbed, molecules must overcome a different resistance to penetration in each type of tissue. Transport across the skin membrane is thus a complex phenomenon. However, it is the cells of the stratum corneum, which present the primary barrier to absorption of topical compositions or transdermally administered active agents. The stratum corneum is a thin layer of dense, highly keratinized cells approximately 10-15 microns thick over most of the body. It is believed to be the high degree of keratinization within these cells as well as their dense packing which creates in most cases a substantially impermeable barrier to active agent penetration. With many active agents, the rate of permeation through the skin is extremely low without the use of some means to enhance the permeability of the skin.

Numerous chemical agents, i.e., chemical penetration enhancers, have been studied as a means of increasing the rate at which an active agent penetrates through the skin. As will be appreciated by those in the field, chemical penetration enhancers are compounds that are selected to increase the permeability of the stratum corneum, and are incorporated into a transdermally administered formulation and/or used to pretreat the skin in the region to which the transdermal system is to be applied. Ideally, such chemical penetration enhancers or “permeation enhancers,” as the compounds are referred to herein, are compounds that are innocuous and serve merely to facilitate diffusion of the active agent through the stratum corneum and thus enhance the transdermal “flux” of the active agent, i.e., the rate at which the active agent passes through the stratum corneum. Permeation enhancers can be used to enhance the penetration of active agents with diverse physicochemical characteristics.

In the field of transdermal active agent delivery, however, there are limitations on the amount of an active agent/enhancer solution that can be loaded into a monolithic delivery system (i.e., a system in which a single polymeric matrix serves as both the active agent reservoir and the means for affixing the system to the skin) without compromising adhesive properties such as tack, adhesive strength, and overall cohesiveness. Additionally, a substantial increase in loading can reduce the ability of the adhesive matrix to maintain structural integrity and uniformity, resulting in phase separation and migration of some components to the edges of the system. High active agent and solution loading can also cause the adhesive matrix to lose cohesive strength and exhibit low creep resistance at room temperature, which is also called “cold flow.” Systems exhibiting cold flow may leave a significant amount of adhesive residue on the skin when the patch is removed.

In U.S. Pat. No. 6,586,000 to Luo, U.S. Pat. No. 6,558,695 to Luo, U.S. Pat. No. 6,565,879 to Luo, International Patent Publication No. WO 01/43775, all of common assignment herewith to Dermatrends, Inc. (San Diego, Calif.), basic permeation enhancers such as inorganic hydroxides have been disclosed as surprisingly effective in increasing the rate at which even high molecular weight active agents permeate into and through the skin without resulting in skin damage or systemic toxicity. The present invention is directed to an improved transdermal delivery system that addresses the aforementioned problems in the art, and is particularly suited to use with basic enhancers.

### DISCLOSURE OF THE INVENTION

In one embodiment, a transdermal delivery system is provided for administering an active agent through the body surface, comprising:

(a) a polymeric matrix that serves as both an active agent reservoir and a skin contact adhesive layer, wherein the matrix comprises a substantially homogeneous mixture of a polyisobutylene rubber and an insoluble hydrophilic polymer in the form of a powder having a particle size in the range of approximately 1 micron to 300 microns;

(b) a pharmaceutical formulation absorbed in the polymeric matrix, which comprises a therapeutically effective amount of the active agent, an effective flux-enhancing amount of a basic permeation enhancing composition, and a pharmaceutically acceptable aqueous vehicle; and

(c) a backing layer laminated to the polymeric matrix that serves as the outer surface of the device use.

It has now been found that a polymeric active agent reservoir fabricated from an admixture of polyisobutylene and an insoluble hydrophilic polymer in powdered form provides numerous advantages in the transdermal delivery of active agents using basic enhancer compositions. For example, the systems of the invention provide for (1) increased permeation of the active agent through the skin, (2) an improved capability of extracting the active agent and enhancer from the transdermal systems, (3) enhanced structural integrity, (4) good chemical stability, (5) reduced phase separation, and (6) decreased cold flow.

In other embodiments of the invention, a method for using the transdermal delivery systems for administering an active agent is provided, as is the polymeric matrix that serves as the active agent reservoir in the above-described transdermal system.

### BRIEF DESCRIPTION OF THE DRAWINGS

FIG. 1 illustrates the cumulative amount of diclofenac sodium that permeated through skin in vitro from transdermal diclofenac systems prepared and evaluated as described in Example 1.

FIG. 2 illustrates the cumulative amount of meloxicam that permeated through skin in vitro from transdermal diclofenac systems prepared and evaluated as described in Example 2.

FIG. 3 illustrates the cumulative amount of testosterone that permeated through skin in vitro from transdermal testosterone systems prepared and evaluated as described in Example 3.

### DETAILED DESCRIPTION OF THE INVENTION

It is to be understood that the terminology used herein is for the purpose of describing particular embodiments only, and is not intended to be limiting. In addition, as used in this specification and the appended claims, the singular forms “a,” “an,” and “the” include plural referents unless the context clearly dictates otherwise. Thus, for example, reference to “an active agent” includes not only a single active agent but also two or more active agents, reference to “a basic permeation enhancer” includes a single such enhancer as well as two or more such enhancers, reference to “a polymeric powder” includes a single such powder as well as two or more such powders, and the like.

The term “active agent” refers to any agent that is capable of producing a beneficial biological effect, preferably a pharmacological response, which may be therapeutic, diagnostic, or prophylactic in nature. The term also encompasses pharmaceutically acceptable, pharmacologically active derivatives of those active agents specifically mentioned herein, including, but not limited to, salts, esters, amides, proactive agents, active metabolites, isomers, fragments, analogs, and the like. When the term “active agent” is used, then, or when a particular active agent is specifically identified, it is to be understood that pharmaceutically acceptable, pharmacologically active salts, esters, amides, proactive agents, active metabolites, isomers, fragments, analogs, etc. of the active agent are intended as well as the active agent per se. It should be noted that the present systems and methods may be used to administer a single active agent or two or more active agents in combination.

By a “therapeutically effective amount” of an active agent is meant a nontoxic but sufficient amount of the active agent to provide the desired beneficial effect. For example, a therapeutically effective amount of an active agent intended to treat an individual afflicted with a disorder, disease, or other adverse physiological condition is an amount that will effect a reduction in severity and/or frequency of symptoms, eliminate the symptoms and/or their underlying cause, and/or facilitate improvement or remediation of damage. As another example, a therapeutically effective amount of an active agent given to a clinically asymptomatic individual who is susceptible to a particular disorder, disease, or other adverse physiological condition, is an amount that will prevent or minimize the occurrence of symptoms and/or their underlying cause.

The amount of active agent that is “therapeutically effective” will vary from subject to subject, depending on the age and general condition of the individual, the particular active agent or agents, and the like. Thus, it is not always possible to specify an exact “effective amount.” However, an appropriate “effective” amount in any individual case may be determined by one of ordinary skill in the art using routine experimentation. When the term “therapeutic effective amount” is used to refer to an amount in a formulation that is not in a finite dosage form, e.g., a formulation that is a gel, cream, lotion, paste, or the like, the term refers to a concentration of the active agent in the formulation that corresponds to a therapeutically effective amount of the active agent in a unit dosage of the formulation.

By “pharmaceutically acceptable,” such as in the recitation of a “pharmaceutically acceptable carrier” or a “pharmaceutically acceptable additive,” is meant a material that is not biologically or otherwise undesirable, i.e., the material may be incorporated into a pharmaceutical formulation or delivery system of the invention without causing any appreciable biological effects or interacting in a deleterious manner with any of the other components of the formulation or system in which it is contained. “Pharmacologically active,” as in a “pharmacologically active” derivative of an active agent, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term “pharmaceutically acceptable” is used to refer to a carrier or excipient, it implies that the excipient has met the standards of toxicological and manufacturing testing required by the U.S. Food & Active agent Administration for inactive ingredients.

The term “aqueous” as applied to a formulation of the invention is used to indicate that the formulation contains water or becomes water-containing following application to the skin or mucosal tissue.

“Penetration enhancement” or “permeation enhancement” as used herein relates to an increase in the permeability of the skin or mucosal tissue to the selected pharmacologically active agent so that the rate at which the agent permeates therethrough, i.e., the “flux” of the agent through the body surface is increased relative to the rate that would be obtained in the absence of permeation enhancer. The enhanced permeation effected through the use of such enhancers can be observed by measuring the rate of diffusion of active agent through animal or human skin using, for example a Franz diffusion apparatus as known in the art and as employed in the Examples herein.

An “effective permeation enhancing amount” of a permeation enhancer composition of the invention refers to a nontoxic, non-damaging but sufficient amount of the enhancer composition to provide the desired increase in flux of an active agent through human skin or mucosal tissue, and, correspondingly, the desired depth of penetration, rate of administration, and amount of active agent delivered.

A “localized region” of skin or mucosal tissue refers to the area of an individual's body surface through which an active agent-enhancer formulation is delivered, and is a defined area of intact unbroken living skin or mucosal tissue. That area will usually be in the range of approximately 5 to approximately 200 cm², more usually in the range of approximately 5 to approximately 100 cm², preferably in the range of approximately 10 to approximately 90 cm², more preferably in the range of approximately 15 to approximately 80 cm², and most preferably in the range of approximately 20 to approximately 60 cm². However, it will be appreciated by those skilled in the art of active agent delivery that the area of skin or mucosal tissue through which active agent is administered may vary significantly, depending on the nature of the formulation, the particular active agent administered, the intended dose, the patch configuration, and the like.

“Transdermal” delivery refers to the administration of an active agent to the skin surface of an individual so that the active agent passes through the skin tissue and into the individual's blood stream, thereby providing a systemic effect. The term “transdermal” is intended to include “transmucosal” administration, i.e., administration of an active agent to a mucosal (e.g., sublingual, buccal, nasal, vaginal, rectal) surface within an individual's body so that the active agent passes through the mucosal tissue and into the blood stream. Correspondingly, the term “skin” as used herein includes mucosal surfaces.

Accordingly, in one embodiment, the invention provides a transdermal delivery system that contains a polymeric matrix which serves as an active agent reservoir. The transdermal systems will generally although not necessarily be “monolithic,” meaning that the polymeric matrix doubles as the active agent reservoir and the skin contact adhesive layer, and that the system, in a preferred embodiment, does not contain any additional layers other than a backing (and, prior to use, a release liner). The polymeric matrix is composed of a mixture of a polyisobutylene (PIB) rubber and an insoluble hydrophilic polymer in the form of a powder. A pharmaceutical formulation is absorbed in the polymeric matrix which contains a number of components, most or all of which are in solution: a therapeutically effective amount of an active agent, an effective flux-enhancing amount of a basic permeation enhancing composition, and a pharmaceutically acceptable aqueous vehicle.

Various steps may be taken in order to ensure that the skin-contacting face of the transdermal system has sufficient tack to releasably adhere to the skin during active agent administration. For example, a lower molecular weight PIB may be employed, and/or a mixture of PIBs that includes a lower molecular weight PIB. Alternatively, or in addition, the PIB may be admixed with an additive that serves to increase the tack of the exposed face of the skin-contacting adhesive layer. Polybutenes are particularly suitable such additives. An ideal polybutene-modified PIB for use in the present systems is that available commercially under the tradename Duro-Tak® 87-6430 from National Starch & Chemical Co.

Another option is to incorporate a water-swellable polymer into the matrix in an amount sufficient to increase the tack of the exposed face of the skin-contacting adhesive layer. Water-swellable polymers, by definition, are those that are capable of absorbing water and physically swell as a result. Suitable water-swellable polymers for use herein may be synthetic, semi-synthetic or naturally occurring, and may be linear or branched. Such polymers include, by way of example: polyalkylene oxides, particularly poly(ethylene oxide) (PEO) and poly(ethylene oxide)-poly(propylene oxide) copolymers; acrylic acid and methacrylic acid polymers, copolymers and esters thereof, preferably formed from acrylic acid, methacrylic acid, methyl acrylate, ethyl acrylate, methyl methacrylate, ethyl methacrylate, and copolymers thereof, with each other or with additional acrylate species such as aminoethyl acrylate (e.g., carbomers); cellulosic polymers such as hydroxymethylcellulose, hydroxypropylcellulose, hydroxyethylcellulose, hydroxypropyl methylcellulose, methylcellulose, ethylcellulose, cellulose acetate, cellulose acetate phthalate, cellulose acetate trimellitate, hydroxypropyl methylcellulose phthalate, hydroxypropylcellulose phthalate, cellulose hexahydrophthalate, cellulose acetate hexahydrophthalate, carboxymethylcellulose, carboxymethylcellulose sodium, and microcrystalline cellulose; and other polysaccharides. Preferred water-swellable polymers are polyalkylene oxides, particularly PEO, such as those of the Polyox® family of polymers manufactured by Union Carbide Chemicals and Plastics Company Inc. of Danbury, Conn., USA.

The insoluble hydrophilic polymer in powder form has a particle size in the range of approximately 1 micron to 300 microns, preferably in the range of approximately 10 to approximately 200 microns, more preferably in the range of approximately 20 to approximately 150 microns, and optimally in the range of approximately 40 to approximately 90 microns. The bulk density of the polymer is typically, although not necessarily, in the range of approximately 0.1 g/cm³ to 0.5 g/cm³, and more commonly in the range of approximately 0.2 g/cm³ to 0.4 g/cm³. The polymer may be crosslinked or uncrosslinked, or may comprise an admixture of crosslinked and uncrosslinked polymer. Generally, a lightly crosslinked polymer is preferred herein. Representative examples of these insoluble hydrophilic polymers include, without limitation, polyvinylpolypyrrolidone (PVPP), sodium carboxymethylcellulose, sodium polyacrylates, and starch, poly(acrylamide-acrylic acid) sodium salt.

The polymeric matrix may also contain an emulsifier to increase the chemical and physical compatibility of certain matrix components and thus provide for a substantially homogeneous admixture, particularly of the hydrophobic PIB and the insoluble hydrophilic polymer. Ideal emulsifiers are compounds having a hydrophobic region, e.g., a long-chain alkyl group, as well as a polar or ionized group, e.g., a hydroxyl group, a carboxylic acid group, a carboxylate, etc. Such compounds include, by way of example, fatty acids and fatty alcohols.

Turning now to the pharmaceutical formulation contained within the aforementioned polymer matrix, the basic permeation enhancer composition, first of all, is composed of at least one pharmaceutically acceptable base, and may be either inorganic or organic. The pH at the body surface-delivery system interface is the primary design consideration, i.e., the delivery system is designed so as to provide the desired pH at the interface. Accordingly, the pH of the pharmaceutical formulation within the polymeric matrix will generally be in the range of approximately 8.0 to approximately 13.0, preferably approximately 8.0 to approximately 11.5, more preferably approximately 8.5 to approximately 11.5, preferably approximately 8.5 to approximately 10.5, more preferably approximately 9.0 to approximately 10.5, and most preferably 9.0-10.0. Suitable bases include inorganic hydroxides, inorganic oxides, inorganic salts of weak acids, nitrogenous bases, and combinations thereof. Inorganic hydroxides are preferred, as are combinations of inorganic hydroxides and nitrogenous bases.

Inorganic hydroxides are generally selected from alkali metal hydroxides, alkaline earth metal hydroxides, ammonium hydroxide, and combinations thereof. Alkali metal hydroxides include sodium hydroxide and potassium hydroxide, while alkaline earth metal hydroxides include calcium hydroxide and magnesium hydroxide. Preferred inorganic hydroxides are alkali metal hydroxides, particularly sodium hydroxide. As indicated in the table below, a 0.1M aqueous solution of an alkali metal hydroxide has a pH of approximately 13.0 when measured at 25° C. When an inorganic hydroxide is used as the sole component of the basic permeation enhancer composition, the amount of inorganic hydroxide is generally in the range of approximately 0.3 wt. % to approximately 7.0 wt %, preferably approximately 0.5 wt. % to approximately 4.0 wt %, more preferably approximately 0.5 wt. % to approximately 3.0 wt %, most preferably approximately 0.75 wt. % to approximately 2.0 wt %, of the formulation present in the polymeric reservoir. The aforementioned amounts are particularly applicable to those transdermal delivery systems in which: (1) the active agent is an uncharged molecule, e.g., a basic active agent is in electronically neutral form; (2) the active agent is in the form of a basic addition salt of an acidic agent; and/or (3) there are no additional species in the formulation or patch that could react with or be neutralized by the inorganic hydroxide, to any significant degree.

For systems in which the active agent is in the form of an acid addition salt, and/or wherein there are additional species in the formulations or systems that can be neutralized by or react with the base (i.e., inactive acidic components), the amount of inorganic hydroxide is preferably the total of (1) the amount necessary to neutralize the acid addition salt and/or other base-neutralizable species (i.e., the “acidic species”), plus (2) 0.3 wt. % to approximately 7.0 wt %, preferably approximately 0.5 wt. % to approximately 4.0 wt %, more preferably approximately 0.5 wt. % to approximately 3.0 wt %, most preferably approximately 0.75 wt. % to approximately 2.0 wt %, of the formulation present in the adhesive reservoir. That is, for an acid addition salt, the enhancer is preferably present in an amount just sufficient to neutralize the salt, plus an additional amount as in (2) to enhance the flux of the active agent through the skin or mucosal tissue.

Inorganic oxides include, for example, magnesium oxide, calcium oxide, and the like, while inorganic salts of weak acids include, without limitation, dibasic ammonium phosphate, sodium acetate, sodium borate, sodium metaborate, sodium carbonate, sodium bicarbonate, tribasic sodium phosphate, dibasic sodium phosphate, potassium carbonate, potassium bicarbonate, potassium citrate, potassium acetate, dibasic potassium phosphate, tribasic potassium phosphate, magnesium phosphate, and calcium phosphate. With inorganic oxides and inorganic salts of weak acids, the amount incorporated into the present delivery systems may be substantially higher than that set forth above for inorganic hydroxides, and may be as high as 20 wt %, in some cases as high as 25 wt % or higher, but will generally be in the range of approximately 2-20 wt %. As above, these amounts may be adjusted to take into consideration the presence of any base-neutralizable species.

In a particularly preferred embodiment, the basic permeation enhancer composition contains an admixture of an inorganic hydroxide and a nitrogenous base, wherein a 0.1M aqueous solution of the nitrogenous base has a pH that is approximately 1.0 to approximately 6.5 lower than a 0.1 M aqueous solution of the inorganic hydroxide, and preferably approximately 1.5 to approximately 6.5 lower than the pH of a 0.1M aqueous solution of the inorganic hydroxide. In addition, the molar ratio of the nitrogenous base to the inorganic hydroxide in the enhancer composition is in the range of approximately 0.5n:1 to approximately 20n:1, where n is the number of hydroxide ions per molecule of the inorganic hydroxide. Thus, for ammonium hydroxide or an alkali metal hydroxide such as sodium hydroxide, n is 1 and the molar ratio of the nitrogenous base to the inorganic hydroxide is therefore in the range of approximately 0.5:1 to approximately 20:1. For an alkaline earth metal hydroxide such as calcium hydroxide, n is 2 and the molar ratio of the nitrogenous base to the inorganic hydroxide is thus in the range of approximately 1:1 to approximately 40:1. Preferably, the molar ratio of the nitrogenous base to the inorganic hydroxide in the enhancer composition is in the range of approximately 0.5n:1 to approximately 10n:1. It will be appreciated that stronger and/or higher molecular weight nitrogenous bases will be used in lesser quantities, while relatively weak and/or lower molecular weight nitrogenous bases will be used in greater quantities. The increase in the degree of enhancement is far higher than would be expected upon combining the two types of bases in a single formulation or delivery system. In addition, the pH of the system is maintained at an elevated level for a longer time period than possible with prior systems containing only an inorganic hydroxide as a permeation enhancer. This in turn ensures that with a hydrophilic active agent whose aqueous solubility decreases with decreasing pH (typically acidic active agents), the active agent will be delivered over an extended time period without precipitation. This “combination” enhancer composition will typically represent approximately 0.3 wt. % to approximately 7.0 wt. %, preferably approximately 0.5 wt. % to approximately 4.0 wt. %, more preferably approximately 0.5 wt. % to approximately 3.0 wt. %, most preferably approximately 0.75 wt. % to approximately 2.0 wt. %, of the polymeric matrix.

Nitrogenous bases include primary amines, secondary amines, tertiary amines, amides, oximes, nitriles, nitrogen-containing heterocycles, and urea. Mixtures of nitrogenous bases can also be used. Preferred nitrogenous bases herein are amino alcohols and urea. Exemplary amino alcohols are those of the formula NR¹R²R³ wherein R¹ is hydroxyl-substituted C₁-C₁₈ hydrocarbyl, and R² and R³ are selected from H, C₁-C₁₈ hydrocarbyl (optionally substituted with a substituent other than hydroxyl), and hydroxyl-substituted C₁-C₁₈ hydrocarbyl. Of these, preferred amino alcohols are those wherein R¹ is C₁-C₁₂ alkyl substituted with 1 to 12 hydroxyl groups, and R² and R³ are selected from H, C₁-C₁₂ alkyl (optionally substituted with substituents other than hydroxyl), and C₁-C₁₂ alkyl substituted with 1 to 12 hydroxyl groups, and more preferred amino alcohols are those wherein R¹ is C₁-C₆ alkyl substituted with 1 to 5 hydroxyl groups, and R² and R³ are selected from H, C₁-C₆ alkyl, and C₁-C₆ alkyl substituted with 1 to 5 hydroxyl groups. Specific examples of the more preferred amino alcohols, then, are triethanolamine (R¹, R², and R³ are —CH₂CH₂OH), diethanolamine (R¹ and R² are —CH₂CH₂OH, and R³ is H), N-methyl glucamine (R¹ is —CH₂—[CH(OH)]₄—CH₂OH, R² is CH₃, and R³ is H) (also referred to as “meglumine”), 2-amino-2-methyl-1,3 propanediol (R¹ is —C(CH₃)(CH₂OH)₂, and R² and R³ are H), and 2-amino-2-methyl-1-propanol (R¹ is —C(CH₃)₂(CH₂OH), and R² and R³ are H).

Other preferred nitrogenous bases include, without limitation: alkylamines (including mono-, di-, and tri-alkylamines) such as methylamine, ethylamine, isopropylamine, n-butylamine, 2-aminoheptane, cyclohexylamine, ethylenediamine, and 1,4-butanediamine; arylamines and aralkylamines such as aniline, N,N-diethylaniline, benzylamine, α-methylbenzylamine, and phenethylamine; aromatic nitrogen-containing heterocycles such as 2-amino-pyridine, benzimidazole, 2,5-diaminopyridine, 2,4-dimethylimidazole, 2,3-dimethylpyridine, 2,4-dimethylpyridine, 3,5-dimethylpyridine, imidazole, methoxypyridine, γ-picoline, and 2,4,6-trimethylpyridine; and non-aromatic nitrogen-containing heterocycles such as 1,2-dimethylpiperidine, 2,5-dimethylpiperazine, 1,2-dimethylpyrrolidine, 1-ethylpiperidine, n-methylpyrrolidine, morpholine, and piperazine.

The strengths of representative bases useful in conjunction with the invention are as follows:

| Base | pH of 0.1 M aqueous sol'n | pKₐ | pK_{b} |
|---|---|---|---|
| sodium hydroxide | 13.0 | — | — |
| potassium hydroxide | 13.0 | — | — |
| triethanolamine | 10.38 | 7.78 | 6.24 |
| diethanolamine | 10.94 | 8.88 | 5.12 |
| N-methyl glucamine | 11.26 | 9.52 | 4.48 |
| urea | 6.55 | 0.10 | 13.9 |
| methylamine | 11.82 | 10.66 | 3.34 |
| ethylamine | 11.90 | 10.81 | 3.19 |
| isopropylamine | 11.80 | 10.60 | 3.40 |
| n-butylamine | 11.81 | 10.61 | 3.39 |
| 2-aminoheptane | 11.85 | 10.70 | 3.30 |
| cyclohexylamine | 11.82 | 10.64 | 3.36 |
| ethylenediamine | 11.46 | 9.93 | 4.07 |
| 1,4-butanediamine | 11.90 | 10.80 | 3.20 |
| aniline | 8.82 | 4.63 | 9.37 |
| N,N-diethylaniline | 7.81 | 2.61 | 11.39 |
| benzylamine | 11.17 | 9.33 | 4.67 |
| α-methyl-benzylamine | 11.38 | 9.75 | 4.25 |
| phenethylamine | 11.42 | 9.83 | 4.17 |
| 2-aminopyridine | 9.91 | 6.82 | 7.18 |
| benzimidazole | 9.24 | 5.48 | 8.52 |
| 2,5-diaminopyridine | 9.74 | 6.48 | 7.52 |
| 2,4-dimethylimidazole | 10.68 | 8.36 | 5.64 |
| 2,3-dimethylpyridine | 9.79 | 6.57 | 7.43 |
| 2,4-dimethylpyridine | 10.00 | 6.99 | 7.01 |
| 3,5-dimethylpyridine | 9.58 | 6.15 | 7.85 |
| imidazole | 9.96 | 6.92 | 7.08 |
| 2-methoxypyridine | 8.14 | 3.28 | 10.72 |
| 3-methoxypyridine | 8.89 | 4.78 | 9.22 |
| 4-methoxypyridine | 9.79 | 6.58 | 7.42 |
| γ-picoline | 9.50 | 5.99 | 8.01 |
| 2,4,6-trimethylpyridine | 9.85 | 6.69 | 7.31 |
| 1,2-dimethylpiperidine | 11.61 | 10.22 | 3.78 |
| 2,5-dimethylpiperazine | 11.33 | 9.66 | 4.34 |
| 1,2-dimethylpyrrolidine | 11.60 | 10.20 | 3.80 |
| 1-ethylpiperidine | 11.73 | 10.45 | 3.55 |
| N-methylpyrrolidine | 11.73 | 10.46 | 3.54 |
| morpholine | 10.75 | 8.50 | 5.50 |
| piperazine | 11.42 | 9.83 | 4.17 |

The active agent administered using the present delivery systems may be any compound that is suitable for transdermal delivery and induces a desired local or systemic beneficial effect. Such compounds include the broad classes of compounds normally delivered through body surfaces and membranes, including skin. While appreciating the fact that active agents may be classified in more than one category, exemplary categories of interest include, without limitation, analgesic agents, anesthetic agents, anti-anginal agents, antiarthritic agents, anti-arrhythmic agents, antiasthmatic agents, antibiotic agents, anticancer agents, anticholinergic agents, anticoagulants, anticonvulsants, antidepressants, antidiabetic agents, antifungal agents, antiglaucoma agents, antigout agents, antihelminthic agents, antihistamines, antihyperlipidemic agents, antihypertensive agents, antiinflammatory agents, antimalarial agents, antimigraine agents, antimuscarinic agents, antinauseants, anti-obesity agents, anti-osteoporosis agents, antipanic agents; antiparkinsonism agents, antiprotozoal agents, antipruritics, antipsychotic agents, antipyretics, antitubercular agents, antitussive agents, antiulcer agents, antiviral agents, anxiolytics, appetite suppressants, calcium channel blockers, cardiac inotropic agents, beta-blockers, bone density regulators, central nervous system stimulants, cognition enhancers, corticosteroids, decongestants, diuretics, gastrointestinal agents, genetic materials, hormonolytics, hypnotics, hypoglycemic agents, immunosuppressants, keratolytics, leukotriene inhibitors, macrolides, mitotic inhibitors, muscle relaxants, narcotic antagonists, neuroleptic agents, nicotine, parasympatholytic agents, peptides, polypeptides, proteins, saccharides, sedatives, sex hormones, sympathomimetic agents, tocolytics, tranquilizers, vasodilators, vitamins, and combinations thereof.

In one preferred embodiment, the active agent is an antiinflammatory agent, generally a nonsteroidal antiinflammatory agent (NSAID) or COX-2 inhibitor. Specific examples of such active agents include, without limitation, acetylsalicylic acid, alclofenac, alminoprofen, benoxaprofen, butibufen, bucloxic acid, carprofen, celecoxib, clidenac, diclofenac, diflunisal, etodolac, fenbufen, fenoprofen, fentiazic, flufenamic acid, flufenasol, flurbiprofen, furofenac, ibufenac, ibuprofen, indomethacin, indoprofen, isoxepac, isoxicam, ketoprofen, ketorolac, meclofenamic acid, mefenamic acid, meloxicam, miroprofen, naproxen, oxaprozin, oxyphenbutazone, oxpinac, parecoxib, phenylbutazone, piclamilast, piroxicam, pirprofen, pranoprofen, rofecoxib, sudoxicam, sulindac, suprofen, tenclofenac, tiaprofenic acid, tolfenamic acid, tolmetin, tramadol, valdecoxib, zomepirac, and pharmacologically active basic addition salts thereof.

In another preferred embodiment, the active agent is a bisphosphonic acid derivative useful in the diagnosis and treatment of disorders and conditions related to bone resorption, calcium metabolism, and phosphate metabolism. Examples of these bisphosphonic acids include 1-hydroxyethane-1,1-diphosphonic acid (etidronic acid), 1,1-dichloromethylene-1,1-bisphosphonic acid (clodronic acid), 3-amino-1-hydroxypropylidene-1,1-bisphosphonic acid (pamidronic acid), 4-amino-1-hydroxybutylidene-1,1-bisphosphonic acid (alendronic acid), 6-amino-1-hydroxyhexylidene-1,1-bisphosphonic acid (neridronic acid), (4-chlorophenyl)thiomethane-1,1-diphosphonic acid (tiludronic acid), 1-hydroxy-2-(3-pyridinyl)-ethylidene-1,1-bisphosphonic acid (risedronic acid), cycloheptylaminomethylene-1,1-bisphosphonic acid (cimadronic acid), 1-hydroxy-3-(N-methyl-N-pentylamino) propylidene-1,1-bisphosphonic acid (ibandronic acid), 3-(dimethylamino)-1-hydroxypropylidene-1,1-bisphosphonic acid (olpadronic acid), [2-(2-pyridinyl)ethylidene]-1,1-bisphosphonic acid (piridronic acid) and 1-hydroxy-2-(1H-imidazol-1-yl)ethylidene-1,1-bisphosphonic acid (zoledronic acid).

In another preferred embodiment, the active agent is a steroidal agent. Steroids include both the corticosteroids and sex hormones; the latter include estrogens, progestins, and androgens.

Estrogens that may be administered using the delivery systems of the invention include synthetic and natural estrogens such as: estradiol (i.e., 1,3,5-estratriene-3,17β-diol, or “17β-estradiol”) and its esters, including estradiol benzoate, valerate, cypionate, heptanoate, decanoate, acetate and diacetate; 17α-estradiol; ethinylestradiol (i.e., 17α-ethinylestradiol) and esters and ethers thereof, including ethinylestradiol 3-acetate and ethinylestradiol 3-benzoate; estriol and estriol succinate; polyestrol phosphate; estrone and its esters and derivatives, including estrone acetate, estrone sulfate, and piperazine estrone sulfate; quinestrol; mestranol; and conjugated equine estrogens. 17β-estradiol, ethinylestradiol and mestranol are particularly preferred synthetic estrogenic agents for use in conjunction with the present invention. Progestins that can be delivered using the systems of the invention include, but are not limited to, acetoxypregnenolone, allylestrenol, anagestone acetate, chlormadinone acetate, cyproterone, cyproterone acetate, desogestrel, dihydrogesterone, dimethisterone, ethisterone (17α-ethinyltestosterone), ethynodiol diacetate, flurogestone acetate, gestadene, hydroxyprogesterone, hydroxyprogesterone acetate, hydroxyprogesterone caproate, hydroxymethylprogesterone, hydroxymethylprogestetone acetate, 3-ketodesogestrel, levonorgestrel, lynestrenol, medrogestone, medroxyprogesterone acetate, megestrol, megestrol acetate, melengestrol acetate, norethindrone, norethindrone acetate, norethisterone, norethisterone acetate, norethynodrel, norgestimate, norgestrel, norgestrienone, nornethisterone, and progesterone. Progesterone, medroxyprogesterone, norethindrone, norethynodrel, d,1-norgestrel and 1-norgestrel are particularly preferred progestins. It is generally desirable to co-administer a progestin along with an estrogen in female HRT so that the estrogen is not “unopposed.” As is well known, estrogen-based therapies are known to increase the risk of endometrial hyperplasia and cancer, as well as the risk of breast cancer, in treated individuals. Co-administration of estrogenic agents with a progestin has been found to decrease the aforementioned risks. Preferred such combinations include, without limitation: 17β-estradiol and medroxyprogesterone acetate; 17β-estradiol and norethindrone; 17β-estradiol and norethynodrel; ethinyl estradiol and d,1-norgestrel; ethinyl estradiol and 1-norgestrel; and megestrol and medroxyprogesterone acetate. For female HRT, it may be desirable to co-administer a small amount of an androgenic agent along with the progestin and the estrogen, in order to reproduce the complete hormone profile of the premenopausal woman, since low levels of certain androgens are present in premenopausal women.

Androgenic agents that may be administered using the delivery systems of the present invention include, but are not limited to: the naturally occurring androgens and derivatives thereof, including androsterone, androsterone acetate, androsterone propionate, androsterone benzoate, androstenediol, androstenediol-3-acetate, androstenediol-17-acetate, androstenediol-3,17-diacetate, androstenediol-17-benzoate, androstenediol-3-acetate-17-benzoate, androstenedione, dehydroepiandrosterone (DHEA; also termed “prasterone”), sodium dehydroepiandrosterone sulfate, 4-dihydrotestosterone (DHT; also termed “stanolone”), dromostanolone, dromostanolone propionate, ethylestrenol, nandrolone phenpropionate, nandrolone decanoate, nandrolone furylpropionate, nandrolone cyclohexanepropionate, nandrolone benzoate, nandrolone cyclohexanecarboxylate, oxandrolone, stanozolol and testosterone; pharmaceutically acceptable esters of testosterone and 4-dihydrotestosterone, typically esters formed from the hydroxyl group present at the C-17 position, including, but not limited to, the enanthate, propionate, cypionate, phenylacetate, acetate, isobutyrate, buciclate, heptanoate, decanoate, undecanoate, caprate and isocaprate esters; and pharmaceutically acceptable derivatives of testosterone such as methyl testosterone, testolactone, oxymetholone and fluoxymesterone.

Still other preferred active agents that can be advantageously administered using the methods, compositions, and systems of the invention are “biomolecules,” i.e., organic molecules (whether naturally occurring, recombinantly produced, or chemically synthesized in whole or in part) that are, were, or can be a part of a living organism. Biomolecules encompass, for example, nucleotides, amino acids, and monosaccharides, as well as oligomeric and polymeric species such as oligonucleotides and polynucleotides, peptidic molecules such as oligopeptides, polypeptides and proteins, saccharides such as disaccharides, oligosaccharides, polysaccharides, mucopolysaccharides or peptidoglycans (peptido-polysaccharides), and the like. See U.S. Pat. No. 6,582,724 to Hsu et al. for additional biomolecules that can be transdermally administered using the present delivery systems.

Acidic active agents will generally, although not necessarily, be incorporated into delivery systems and formulations of the invention in the form of a basic addition salt. Basic addition salts of acidic active agents are prepared from the free acid using conventional methodology, involving reaction with a pharmaceutically acceptable base. Such bases include, by way of example, organic bases such as ethylamine, n-butylamine, n-hexylamine, di-isopropylamine, trimethylamine, triethylamine, 2-diethylaminoethanol, lysine, and choline, and inorganic bases such as sodium hydroxide, potassium hydroxide, ammonium hydroxide, and calcium hydroxide.

Representative basic addition salts of hydrophilic active agents include, without limitation, diclofenac sodium, cromolyn sodium, ketorolac tromethamine, tolmetin sodium, meclofenamate sodium, and etidronate sodium. The basic addition salts may also be associated with water molecules and thus in the form of a hydrate; one such example is 4-amino-1-hydroxy-butylidene-1,1-bisphosphonic acid monosodium salt trihydrate, also known as “alendronate.”

The amount of active agent administered will depend on a number of factors and will vary from subject to subject and depend on the particular active agent administered, the particular disorder or condition being treated, the severity of the symptoms, the subject's age, weight, and general condition, and the judgment of the prescribing physician. Other factors, specific to transdermal active agent delivery, include the solubility and permeability of the carrier and adhesive layer in a transdermal delivery device, if one is used, and the period of time for which such a system will be fixed to the skin or other body surface. The minimum amount of active agent is determined by the requirement that sufficient quantities of active agent must be present in a device or composition to maintain the desired rate of release over the given period of application. The maximum amount for safety purposes is determined by the requirement that the quantity of active agent present cannot exceed a rate of release that reaches toxic levels. Generally, the maximum concentration is determined by the amount of agent that can be received in the carrier without producing adverse histological effects such as irritation, an unacceptably high initial pulse of agent into the body, or adverse effects on the characteristics of the delivery device such as the loss of tackiness, viscosity, or deterioration of other properties.

Various additives, known to those skilled in the art, may be included in pharmaceutical formulation. For example, solvents, including relatively small amounts of alcohol, may be used to facilitate the solubilization of certain active agents. Other optional additives include opacifiers, antioxidants, fragrance, colorant, gelling agents, thickening agents, stabilizers, surfactants and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof. Still other components that may be present include solubilizers, additional enhancers, viscosity controlling agents, and the like.

The formulation may also contain irritation-mitigating additives to minimize or eliminate the possibility of skin irritation or skin damage resulting from the active agent, the basic enhancer composition, or other components of the formulation. Suitable irritation-mitigating additives include, for example: α-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritation-mitigating additive, if present, will be incorporated into the formulation at a concentration effective to mitigate irritation or skin damage, typically representing not more than approximately 20 wt. %, preferably not more than approximately 10%, more typically not more than approximately 5 wt. %, of the formulation.

The transdermal delivery system of the invention comprises the above-described polymeric matrix of a pharmaceutically acceptable adhesive material that serves to affix the system during active agent administration. The backing layer, to which the polymeric matrix is laminated, functions as the primary structural element of the transdermal delivery system and provides flexibility and, preferably, occlusivity. The material used for the backing layer should be inert and incapable of absorbing the active agent(s), the basic permeation enhancer composition, or other components of the formulation contained within the system. The backing is preferably comprised of a flexible elastomeric material that serves as a protective covering to prevent loss of active agent and/or vehicle via transmission through the upper surface of the system, and will preferably impart a degree of occlusivity to the system, such that the area of the body surface covered by the system becomes hydrated during use. The material used for the backing layer should permit the device to follow the contours of the skin and be worn comfortably on areas of skin such as at joints or other points of flexure, that are normally subjected to mechanical strain with little or no likelihood of the device disengaging from the skin due to differences in the flexibility or resiliency of the skin and the device. The materials used for the backing layer are either occlusive or permeable, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure preferably includes a release liner. Immediately prior to use, this layer is removed from the device so that the system may be affixed to the skin. The release liner should be made from an active agent/enhancer/vehicle impermeable material, and is a disposable element, which serves only to protect the device prior to application. Typically, the release liner is formed from a material impermeable to the pharmacologically active agent and the base enhancer, and is easily stripped from the transdermal patch prior to use.

Additional layers, e.g., intermediate fabric layers and/or rate-controlling membranes, will not generally be present in these transdermal systems, although they may in certain cases be advantageously included. Fabric layers may be used to facilitate fabrication of the device, while a rate-controlling membrane may be used to control the rate at which a component permeates out of the device. The component may be an active agent, a base enhancer, an additional enhancer, or some other component contained in the transdermal delivery system. A rate-controlling membrane, if present, will be included in the system on the skin side of one or more of the active agent reservoirs. The material used to form such a membrane is selected so as to limit the flux of one or more components contained in the pharmaceutical formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal delivery system, i.e., the skin contact area, has an area in the range of approximately 5 to approximately 200 cm², preferably approximately 5 to approximately 100 cm², preferably in the range of approximately 10 to approximately 90 cm², more preferably in the range of approximately 15 to approximately 80 cm², and most preferably in the range of approximately 20 to approximately 60 cm². That area will vary, of course, with the amount of active agent to be delivered and the flux of the active agent through the body surface. Larger patches can be used to accommodate larger quantities of active agent, while smaller patches can be used for smaller quantities of active agent and/or agents that exhibit a relatively high permeation rate.

The present transdermal delivery systems are fabricated by sequential admixture of components, with heating and agitation or stirring carried out as necessary. The admixture so prepared may be cast, in fluid form, onto the backing layer, followed by lamination of the release liner. Similarly, the admixture may be cast onto the release liner, followed by lamination of the backing layer. Alternatively, but in a less preferred embodiment, the polymeric matrix may be prepared in the absence of active agent or excipient, and then loaded by soaking in an active agent/enhancer composition/vehicle mixture. In general, transdermal systems of the invention are fabricated by solvent evaporation, film casting, melt extrusion, thin film lamination, die cutting, or the like. The basic permeation enhancer composition will generally be incorporated into the transdermal delivery system during patch manufacture rather than subsequent to preparation of the device. Accordingly, the basic permeation enhancer composition will then convert a nonionized acidic active agent to the ionized active agent in salt form.

An adhesive overlayer that also serves as a backing for the delivery system may be used to better secure the patch to the body surface. This overlayer is sized such that it extends beyond the polymeric active agent reservoir so that adhesive on the overlayer comes into contact with the body surface. Such an overlayer may be useful because the adhesive/polymeric matrix layer may lose a certain amount of tack after application due to hydration.

Other types and configurations of transdermal active agent delivery systems may also be used in conjunction with the method of the present invention, as will be appreciated by those skilled in the art of transdermal active agent delivery. See, for example, Ghosh, Transdermal and Topical Active agent Delivery Systems (Interpharm Press, 1997), particularly Chapters 2 and 8.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of active agent formulation, particularly topical and transdermal active agent formulation, which are within the skill of the art. Such techniques are fully explained in the literature. See Remington: The Science and Practice of Pharmacy, cited supra, as well as Goodman & Gilman's The Pharmacological Basis of Therapeutics, 10^{th} Ed.(2001).

The following examples are put forth so as to provide those of ordinary skill in the art with a complete disclosure and description of how to practice the methods as well as make and use the compositions of the invention, and are not intended to limit the scope of what the inventors regard as their invention. Efforts have been made to ensure accuracy with respect to numbers (e.g., amounts, temperature, etc.) but some errors and deviations should be accounted for. Unless indicated otherwise, parts are parts by weight, temperature is in ° C., and pressure is at or near atmospheric.

### EXAMPLE 1

### Transdermal Diclofenac Sodium Systems

An in vitro skin permeation study was conducted using four diclofenac sodium transdermal delivery systems. The components used to prepare each system are listed in Table 1, along with the actual weight of each component and the weight percent (based on total solution weight) in each formulation. The systems were prepared by combining the first six components in Table 1 and heating the admixture to effect dissolution. The insoluble, hydrophilic polymer, either ISP Polyplasdone® XL-10 Crospovidone or starch, poly(co-acrylamide-acrylic acid) sodium salt, was then added, and the mixture was stirred. The polyisobutylene/polybutene adhesive (National Starch & Chemical Co.) and heptane were then added, and the mixture stirred again. Each formulation was coated onto a release liner and dried in an oven at 65° C. for two hours to remove water and other solvents. The dried active agent-in-adhesive/release liner film was laminated to a backing film, and the backing/active agent-in-adhesive/release liner laminate was then cut into discs with a diameter of 9/16 inch.

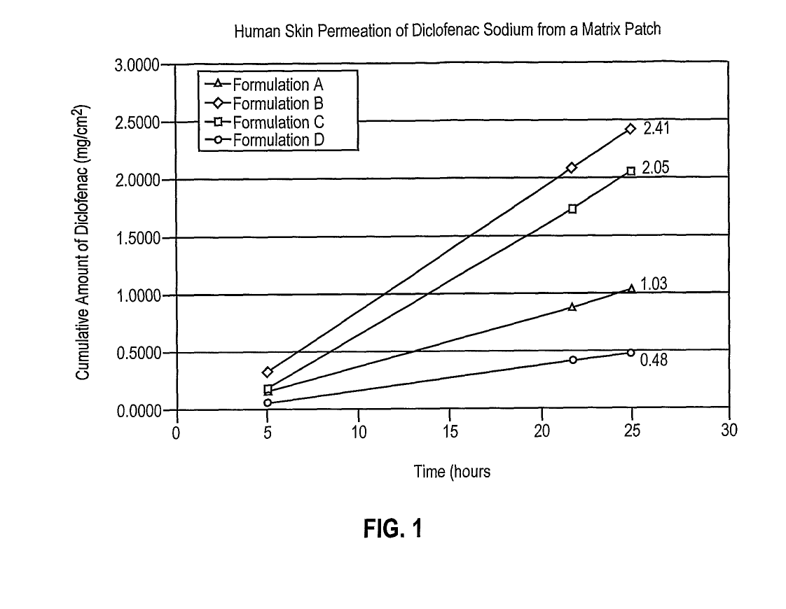

The in vitro permeation of diclofenac sodium through human cadaver skin from these discs was evaluated using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/active agent-in-adhesive film was then pressed onto the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was a 0.05M KH₂PO₄ solution adjusted to pH 6.5. All cells and receiver solution were maintained at 32° C. for the 24-hour duration of the study. The entire receiver solution was collected and replaced with fresh phosphate buffered solution at each time point. The receiver solution collected was analyzed by HPLC to determine the concentration of diclofenac sodium. The cumulative amount of diclofenac sodium that permeated across the human cadaver skin was calculated using the measured diclofenac concentrations in the receiver solutions, and the results were plotted versus time (FIG. 1).

The cumulative amount of diclofenac that permeated through the skin was 0.48 mg/cm² when no PVPP powder was added. When the PVPP powder was added, the cumulative amount of diclofenac that permeated through the skin was 2.41 mg/cm², more than five times the cumulative amount permeated in the absence of the powder. When the PVPP powder was added in combination with polyethylene oxide (PEO), the cumulative amount of diclofenac that permeated through the skin was 2.05 mg/cm², indicating that PEO can be used to decrease skin permeation to a small extent if so desired. When powdered starch (i.e., starch, poly(acrylamide-acrylic acid) sodium salt) was used instead of PVPP, the cumulative amount of diclofenac that permeated through the skin was also increased substantially, to 1.03 mg/cm².

TABLE 1
Weight and Weight Percent of Components (Based on Total Solution Weight)

| | Formulation A | Formulation B | Formulation C | Formulation D |
|---|---|---|---|---|
| Component | wt., g (wt. %) | wt., g (wt. %) | wt., g (wt. %) | wt., g (wt. %) |
| Diclofenac sodium | 0.8 (6.1%) | 1.0 (7.7%) | 1.0 (7.6%) | 1.0 (7.6%) |
| Benzyl alcohol | 0.3 (2.3%) | 0.3 (2.3%) | 0.3 (2.3%) | 0.3 (2.3%) |
| Triethanolamine | 0.4 (3.1%) | 0.4 (3.1%) | 0.4 (3.1%) | 0.4 (3.1%) |
| 1,3-Butanediol | 1.0 (7.7%) | 1.0 (7.7%) | 1.0 (7.6%) | 1.0 (7.6%) |
| Oleic acid | 0.1 (0.8%) | 0.1 (0.8%) | 0.1 (0.8%) | 0.1 (0.7%) |
| N-methyl glucamine | 0.35 (2.7%) | 0.35 (2.7%) | 0.35 (2.6%) | 0.35 (2.3%) |
| Crospovidone | — | 0.70 g (5.4%) | 0.70 g (5.3%) | — |
| (ISP Polyplasdone XL- | | | | |
| 10) | | | | |
| Starch, poly(acrylamide- | 0.70 g (5.4%) | — | — | — |
| acrylic acid) sodium salt | | | | |
| PIB/polybutene adhesive | 8.1 (62.0%) | 8.1 (62.7%) | 8.1 (61.3%) | 10.6 (70.9%) |
| (Duro-Tak ® 87-6430) | | | | |
| N-heptane | 1.0 (7.7%) | 0.75 (5.8%) | 1.0 g (7.6%) | 1.0 (6.7%) |
| poly(ethylene oxide) | 0.1 (0.8%) | — | 0.05 (0.4%) | — |
| NaOH (1:1 NaOH:H₂O)) | 0.209 (1.6%) | 0.209 (1.6%) | 0.219 (1.7%) | 0.209 (1.4%) |

### Extraction of Diclofenac Sodium from the Transdermal Systems:

Two additional diffusion cells per formulation were set up for purposes of measuring the pH on the surface of the skin and for measurement of diclofenac extraction from the above transdermal systems. After removing the transdermal systems from the cadaver skin five hours following application, the active agent was extracted from the systems using 100% deionized (DI) water. Each extraction was carried out in duplicate.

In order to carry out the extractions, two scintillation vials were used per transdermal system, each containing 10 ml of ultra-pure water. The systems were initially placed in the first vial for 15 seconds, with agitation, to dissolve active agent that adhered to the system surface. The systems were then placed in the second vial and agitated, at which point agitation was stopped but extraction allowed to continue for 19 hours. Prior to filtering the extraction solutions for HPLC analysis, the vials were agitated once more. The amount of active agent extracted from each system was determined, and the results are presented in Table 2.

TABLE 2
Diclofenac Sodium Patch Extractions

| Formulation | Extracted | Mean | % Recovery |
|---|---|---|---|
| A-1 | 4.7 mg | 4.7 ± 0.0 mg | 71% |
| A-2 | 4.6 mg | | |
| B-1 | 5.7 mg | 5.9 ± 0.3 mg | 66% |
| B-2 | 6.1 mg | | |
| C-1 | 5.5 mg | 5.6 ± 0.1 mg | 67% |
| C-2 | 5.7 mg | | |
| D-1 | 1.1 mg | 1.2 ± 0.2 mg | 17% |
| D-2 | 1.3 mg | | |

The results clearly indicate that the inclusion of starch or PVPP powder into the formulations facilitates the extraction of diclofenac as demonstrated by the percent recovery figures in Table 2. As may be seen, the percent of active agent recovered was 4.2 times higher for the system prepared with Formulation A than for the system prepared with Formulation D, which did not contain any PVPP powder.

### Skin Surface pH Measurement After Five Hours in an in vitro Study:

The pH of the skin surface was measured five hours into the skin permeation study described above. The Franz chambers were prepared for measuring skin surface pH by removing the receiving fluid and placing it in a test tube, and removing the clamp and donor chamber. The transdermal system was gently removed from the skin with tweezers, leaving the skin on the receiving chamber. The receiving chamber was placed in the Franz cell to immobilize it, and a microelectrode was placed directly onto the skin surface with sufficient pressure to ensure contact of the electrode tip with the fluid on the skin surface. If the skin surface was completely dry, one or two droplets of DI water were applied to the skin before applying the microelectrode. Using a pH meter calibrated to the correct range, the pH of the skin surface was recorded. The results are set forth in Table 3.

TABLE 3
Skin Surface pH measurement

| Formulation | pH | Mean |
|---|---|---|
| A-1 | 11.60 | 11.55 |
| A-2 | 11.50 | |
| B-1 | 10.84 | 10.77 |
| B-2 | 10.70 | |
| C-1 | 11.00 | 10.43 |
| C-2 | 9.86 | |
| D-1 | 9.60 | 9.55 |
| D-2 | 9.50 | |

Table 3 demonstrates the effectiveness of a powder additive in facilitating the release of basic enhancer from the matrix patch formulation. The only system not containing powder, that prepared with Formulation D, demonstrated a mean pH of 9.55 at the five-hour point, while the other formulations resulted in a mean pH in the range of 10.43 to 11.55.

### EXAMPLE 2

### Transdermal Meloxicam Systems

An in vitro skin permeation study was conducted using four meloxicam transdermal delivery systems. The components used to prepare each system are listed in Table 4, along with the actual weight of each component and the weight percent (based on total solution weight) in each formulation. The systems were prepared by combining the first six components in Table 1 and heating the admixture to effect dissolution. The benzyl alcohol, DI water, and oleic acid were then added, and the mixture stirred. The remaining components, i.e., the polyvinylpolypyrrolidone powder, the adhesive in heptane, and the PEO were then added in sequence, with stirring after addition of each component. Each formulation was coated onto a release liner and dried in an oven at 65° C. for two hours to remove water and other solvents. The dried active agent-in-adhesive/release liner film was laminated to a backing film and stored in a heat-sealed foil pouch to maintain moisture following removal from the oven, until permeation testing. The backing/active agent-in-adhesive/release liner laminate was then cut into discs with a diameter of 9/16 inch.

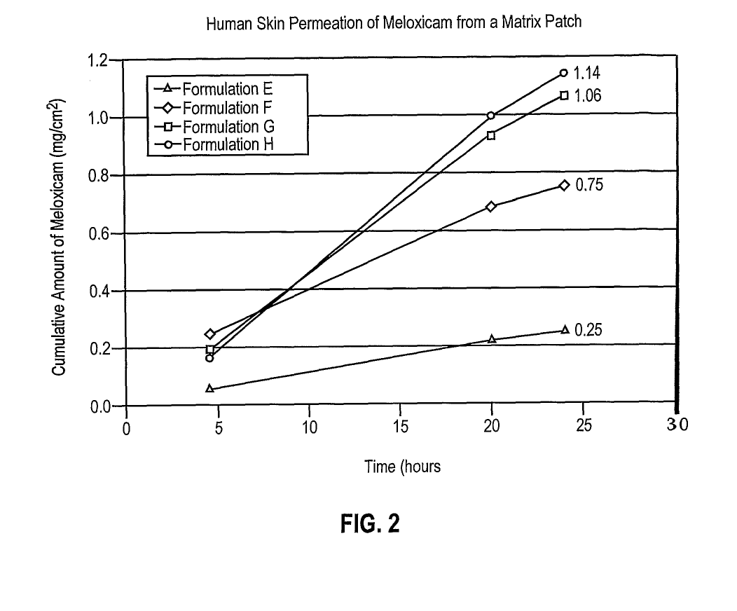

The in vitro permeation of meloxicam through human cadaver skin from these discs was evaluated using the method of Example 1. The cumulative amount of meloxicam that permeated across the human cadaver skin was calculated using the measured meloxicam concentrations in the receiver solutions. The results were plotted versus time (FIG. 2).

The cumulative amount of meloxicam that permeated through the skin was 0.25 mg/cm² when no PVPP powder was added. When 0.23 g of the PVPP powder was added, the cumulative amount of meloxicam that permeated through the skin was 0.75 mg/cm², three times the cumulative amount permeated in the absence of the powder. When 0.47 g of the PVPP powder was added, the cumulative amount of meloxicam that permeated through the skin increased to 1.06 mg/cm², and when 0.70 g PVPP powder was used, the cumulative amount of meloxicam that permeated through the skin increased to 1.14 mg/cm², which was approximately 4.6 times the cumulative amount permeated in the absence of PVPP powder.

TABLE 4
Weight and Weight Percent of Components (Based on Total Solution Weight)

| | Formulation E | | Formulation F | | Formulation G | | Formulation H | |
|---|---|---|---|---|---|---|---|---|
| Component | wt., g/wt. % | | wt., g/wt. % | | wt., g/wt. % | | wt., g/wt. % | |
| Meloxicam | 0.35 | 3.0% | 0.35 | 2.9% | 0.35 | 2.8% | 0.35 | 2.8% |
| Hexylene Glycol | 0.50 | 4.2% | 0.50 | 4.1% | 0.50 | 4.0% | 0.50 | 4.0% |
| Triethanolamine | 0.50 | 4.2% | 0.50 | 4.1% | 0.50 | 4.0% | 0.50 | 4.0% |
| 1,3-butanediol | 0.15 | 1.3% | 0.15 | 1.2% | 0.15 | 1.2% | 0.15 | 1.2% |
| N-methyl glucamine | 0.35 | 3.0% | 0.35 | 2.9% | 0.35 | 2.8% | 0.35 | 2.8% |
| NaOH | 0.144 | 1.2% | 0.144 | 1.2% | 0.144 | 1.2% | 0.144 | 1.1% |
| Benzyl Alcohol | 0.20 | 1.7% | 0.20 | 1.7% | 0.20 | 1.6% | 0.20 | 1.6% |
| DI H₂O | 0.24 | 2.0% | 0.24 | 2.0%% | 0.24 | 1.9% | 0.24 | 1.9% |
| Oleic Acid | 0.05 | 0.4% | 0.05 | 0.4% | 0.05 | 0.4% | 0.05 | 0.4% |
| Polyvinylpolypyrrolidone | — | 0.0% | 0.23 | 1.9% | 0.47 | 3.8% | 0.47 | 5.6% |
| PIB/Polybutene Adhesive | 9.00 | 76.1% | 9.00 | 74.3% | 9.00 | 72.9% | 9.00 | 71.5% |
| n-Heptane | 0.35 | 3.0% | 0.35 | 2.9% | 0.35 | 2.8% | 0.35 | 2.8% |
| Poly(Ethylene Oxide) | — | 0.0% | 0.05 | 0.4% | 0.05 | 0.4% | 0.05 | 0.4% |

### EXAMPLE 3

### Transdermal Testosterone Systems

An in vitro skin permeation study was conducted using two testosterone transdermal delivery systems. The components used to prepare each system are listed in Table 5, along with the actual weight of each component and the weight percent (based on total solution weight) in each formulation. The systems were prepared by combining the first five components in Table 5 and heating the admixture to effect dissolution. The remaining components, i.e., the PVPP powder (if any), the adhesive, the PEO (if any), and the NaOH in water were added in sequence, with stirring after addition of each component. The formulations were coated onto a release liner and discs prepared therefrom as described in Example 2.

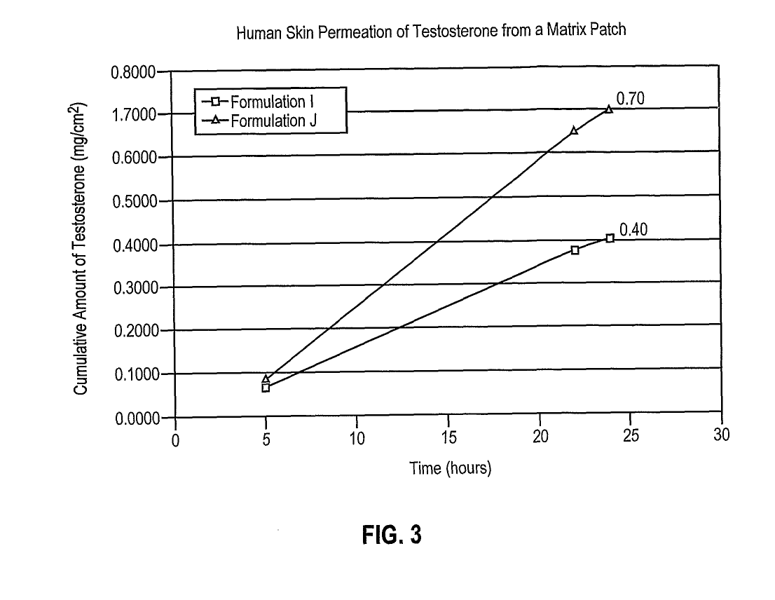

The in vitro permeation of testosterone through human cadaver skin from these discs was evaluated using Franz diffusion cells with a diffusion area of 1 cm² and a receiver solution capacity of 8 ml. Human cadaver skin was cut to a proper size and placed on a flat surface with the stratum corneum side facing up. The release liner was peeled away from the disc laminate. The backing/active agent-in-adhesive film was then pressed onto the skin with the adhesive side facing the stratum corneum. The skin/adhesive/backing laminate was clamped between the donor and receiver chambers of the diffusion cell with the skin side facing the receiver solution. Three diffusion cells were used for each formulation. The receiver solution was 30% N-methyl pyrrolidone (NMP) in 70% DI H₂O (v/v). The entire receiver solution was collected and replaced with fresh 30% NMP solution at each time point. The receiver solution collected was analyzed by HPLC to determine the concentration of testosterone. The cumulative amount of testosterone that permeated across the human cadaver skin was calculated using the measured testosterone concentrations in the receiver solutions, and the results were plotted versus time (FIG. 3).

The cumulative amount of testosterone that permeated through the skin was 0.40 mg/cm² when no PVPP powder was added. When 0.75 g of the PVPP powder was added, the cumulative amount of testosterone that permeated through the skin was 0.70 mg/cm², almost twice the cumulative amount permeated in the absence of the powder.

TABLE 5
Weight and Weight Percent of Components
(Based on Total Solution Weight)

| | | |
|---|---|---|
| | Formulation I | Formulation J |
| Component | wt., g/wt. % | wt., g/wt. % |

| | | | | |
|---|---|---|---|---|
| Testosterone | 0.6 | 3.4% | 0.6 | 3.9% |
| Benzyl Alcohol | 0.3 | 1.7% | 0.3 | 2.0% |
| Hexylene Glycol | 0.7 | 4.0% | 0.7 | 4.6% |
| Igepal ® CO | 0.15 | 0.9% | 0.15 | 1.0% |
| Glycerin | 0.7 | 4.0% | 0.7 | 4.6% |
| Polyvinylpolypyrrolidone | 0 | 0.0% | 0.75 | 4.9% |
| Poly(Ethylene Oxide) | 0 | 0.0% | 0.05 | 0.3% |
| Durotak ® 87-6430 | 15 | 85.3% | 12 | 78.0% |
| NaOH | 0.065 | 0.4% | 0.065 | 0.4% |
| H₂O | 0.065 | 0.4% | 0.065 | 0.4% |

### Extraction of Testosterone from the Transdermal Systems:

Two additional diffusion cells per formulation were set up for purposes of measuring the extraction of testosterone from the above transdermal systems. After removing the transdermal systems from the cadaver skin five hours following application, the active agent was extracted from the systems using a 55:45 (v:v) mixture of ethanol and DI water. Each extraction was carried out in duplicate.

In order to carry out the extractions, two scintillation vials were used per transdermal system, each containing 10 ml of the ethanol/water mixture. The systems were initially placed in the first vial for 15 seconds, with agitation, to dissolve active agent that adhered to the system surface. The systems were then placed in the second vial and agitated, at which point agitation was stopped but extraction allowed to continue for 19 hours. Prior to filtering the extraction solutions for HPLC analysis, the vials were agitated once more. The amount of active agent extracted from each system was determined, and the results are presented in Table 6.

TABLE 6
Testosterone Patch Extractions

| Formulation | Extracted | Mean | % Recovery |
|---|---|---|---|
| I-1 | 1.12 mg | 1.12 ± 0.01 mg | 25% |
| I-2 | 1.11 mg | | |
| J-1 | 3.45 mg | 3.19 ± 0.38 mg | 61% |
| J-2 | 2.92 mg | | |

As in Example 1, the results set forth in Table 6 indicate that the inclusion of the polymer powder into the formulations facilitates the extraction of testosterone, insofar as the amount of active agent recovered was 2.4 times higher for the system prepared with Formulation J than for the system prepared with Formulation I, which did not contain any polymeric powder.

1. A transdermal delivery system for administering an active agent through the body surface, comprising:
(a) a polymeric matrix that serves as both an active agent reservoir and a skin contact adhesive layer, wherein the matrix comprises a substantially homogeneous mixture of a polyisobutylene rubber and an insoluble hydrophilic polymer in the form of a powder having a particle size in the range of about 1 micron to 300 microns;
(b) a pharmaceutical formulation absorbed in the polymeric matrix which comprises a therapeutically effective amount of the active agent, an effective flux-enhancing amount of a basic permeation enhancing composition, and a pharmaceutically acceptable aqueous vehicle; and
(c) a backing layer laminated to the polymeric matrix that serves as the outer surface of the device use.

2. The system of claim 1, wherein the polyisobutylene rubber:
(a) has a molecular weight selected to provide the polymeric matrix with sufficient tack to ensure adhesion of the delivery system to the skin during active agent administration;
(b) is modified by admixture with an additive selected to provide the polymeric matrix with sufficient tack to ensure adhesion of the delivery system to the skin during active agent administration; or
(c) both (a) and (b).

3. The system of claim 2, wherein the additive comprises a polybutene.

4. The system of claim 1, wherein the polymeric matrix further includes at least one of: a water-swellable polymer in an amount sufficient to provide the polymeric matrix with sufficient tack to ensure adhesion of the delivery system to the skin during active agent administration; and an emulsifier.

5. The system of claim 4, wherein the water-swellable polymer is a polyalkylene oxide and the emulsifier is a fatty acid or fatty alcohol.

6. The system of claim 1, wherein the insoluble hydrophilic polymer has a particle size is in the range of about 10 microns to 200 microns.

7. (canceled)

8. (canceled)

9. The system of claim 1, wherein the insoluble hydrophilic polymer has a bulk density in the range of approximately 0.1 g/cm³ to 0.5 g/cm³.

10. The system of claim 9, wherein the insoluble hydrophilic polymer has a bulk density in the range of approximately 0.2 g/cm³ to 0.4 g/cm³.

11. The system of claim 1, wherein the insoluble hydrophilic polymer is lightly crosslinked.

12. The system of claim 1, wherein the insoluble hydrophilic polymer is not crosslinked.

13. The system of claim 1, wherein the insoluble hydrophilic polymer comprises a mixture of crosslinked and uncrosslinked polymer.

14. The system of claim 1, wherein the insoluble hydrophilic polymer is selected from: polyvinylpolypyrrolidone; poly(N-vinyl-2-caprolactam); poly(N-vinyl-2-valerolactam); carboxymethylcellulose sodium; poly(acrylamide-acrylic acid) sodium salt of starch; and sodium polyacrylate.

15-21. (canceled)

22. The system of claim 21, wherein the inorganic hydroxide is selected from ammonium hydroxide, alkali metal hydroxides, alkaline earth metal hydroxides, and combinations thereof, and the nitrogenous base is selected from urea and amino alcohols.

23. The system of claim 22, wherein the inorganic hydroxide is an alkali metal hydroxide and the nitrogenous base is an amino alcohol.

24. The system of claim 23, wherein the amino alcohol is of the structural formula NR¹R²R³ wherein R¹ is hydroxy-substituted hydrocarbyl, and R² and R³ are selected from H, hydrocarbyl, and hydroxy-substituted hydrocarbyl.

25. The system of claim 24, wherein R¹ is C₁-C₁₂ alkyl substituted with 1 to 12 hydroxyl groups, and R² and R³ are selected from H, C₁-C₁₂ alkyl, and C₁-C₁₂ alkyl substituted with 1 to 12 hydroxyl groups.

26. The system of claim 25, wherein R¹ is C₁-C₆ alkyl substituted with 1 to 5 hydroxyl groups, and R² and R³ are selected from H, C₁-C₆ alkyl, and C₁-C₆ alkyl substituted with 1 to 5 hydroxyl groups.

27. The system of claim 26, wherein R¹, R², and R³ are —CH₂CH₂OH, such that the amino alcohol is triethanolamine.

28. The system of claim 26, wherein R¹ and R² are —CH₂CH₂OH, and R³ is H, such that the amino alcohol is diethanolamine.

29. The system of claim 26, wherein R¹ is —CH₂—[CH(OH)]₄ —CH₂OH, R² is CH₃, and R³ is H, such that the amino alcohol is diethanolamine.

30. The system of claim 1, wherein the apparent aqueous solubility of the active agent increases with increasing pH.

31. The system of claim 30, wherein the apparent aqueous solubility of the active agent is greater than 5 mg/ml at a pH of 8.0, when measured at 25° C.

32. The system of claim 31, wherein the apparent aqueous solubility of the active agent is greater than 10 mg/ml at a pH of 9.0, when measured at 25° C.

33. The system of claim 1, wherein the active agent is ionizable.

34. The system of claim 33, wherein the active agent is an acidic agent in the form of a basic addition salt.

35. The system of claim 1, wherein the active agent is selected from analgesic agents, anesthetic agents, anti-anginal agents, antiarthritic agents, anti-arrhythmic agents, antiasthmatic agents, antibiotic agents, anticancer agents, anticholinergic agents, anticoagulants, anticonvulsants, antidepressants, antidiabetic agents, antifungal agents, antiglaucoma agents; antigout agents, antihelminthic agents, antihistamines, antihyperlipidemic agents, antihypertensive agents, antiinflammatory agents, antimalarial agents, antimigraine agents, antimuscarinic agents, antinauseants, anti-obesity agents, anti-osteoporosis agents, antipanic agents; antiparkinsonism agents, antiprotozoal agents, antipruritics, antipsychotic agents, antipyretics, antitubercular agents, antitussive agents, antiulcer agents, antiviral agents, anxiolytics, appetite suppressants, calcium channel blockers, cardiac inotropic agents, beta- blockers, bone density regulators, central nervous system stimulants, cognition enhancers, corticosteroids, decongestants, diuretics, gastrointestinal agents, genetic materials, hormonolytics, hypnotics, hypoglycemic agents, immunosuppressants, keratolytics, leukotriene inhibitors, macrolides, mitotic inhibitors, muscle relaxants, narcotic antagonists, neuroleptic agents, nicotine, parasympatholytic agents, sedatives, sex hormones, sympathomimetic agents, tocolytics, tranquilizers, vasodilators, vitamins, and combinations thereof.

36. The system of claim 1, wherein the backing layer is occlusive.

37. An active agent reservoir comprising:
(a) a polymeric matrix that comprises a substantially homogeneous mixture of a polyisobutylene rubber and an insoluble hydrophilic polymer in the form of a powder having a particle size in the range of approximately 1 micron to 300 microns;
(b) a pharmaceutical formulation absorbed in the polymeric matrix which comprises a therapeutically effective amount of an active agent, an effective flux-enhancing amount of a basic permeation enhancing composition, and a pharmaceutically acceptable aqueous vehicle.

38. A method for transdermally administering an active agent, which comprises affixing a transdermal delivery system to a localized region of a human patient's body surface such that a body surface-delivery system interface is formed, wherein the system comprises:
(a) a polymeric matrix that serves as both an active agent reservoir and a skin contact adhesive layer, wherein the matrix comprises a substantially homogeneous mixture of a polyisobutylene rubber and an insoluble hydrophilic polymer in the form of a powder having a particle size in the range of approximately 1 micron to 300 microns;
(b) a pharmaceutical formulation absorbed in the polymeric matrix which comprises a therapeutically effective amount of the active agent, an effective flux-enhancing amount of a basic permeation enhancing composition, and a pharmaceutically acceptable aqueous vehicle; and
(c) a backing layer laminated to the polymeric matrix that serves as the outer surface of the device use.

39-77. (canceled)
